(19) United States
(12) Patent Application Publication
(10) Pub. No.: US 2017/0311845 A1
(43) Pub. Date: Nov. 2, 2017

(54) METHOD AND APPARATUS FOR DETECTING SMOKING BEHAVIOR BASED ON ACCELERATION SENSOR

(71) Applicant: AJOU UNIVERSITY INDUSTRY-ACADEMIC COOPERATION FOUNDATION, Suwon-si (KR)

(72) Inventors: We Duke CHO, Seongnam-si (KR); Sun Taag CHOE, Suwon-si (KR)

(73) Assignee: AJOU UNIVERSITY INDUSTRY-ACADEMIC COOPERATION FOUNDATION, Suwon-si (KR)

(21) Appl. No.: 15/499,483

(22) Filed: Apr. 27, 2017

(30) Foreign Application Priority Data
Apr. 29, 2016 (KR) 10-2016-0053304

(51) Int. Cl. A61B 5/06; A61B 5/08; A61B 5/00; G06F 3/01; G01N 29/44; A61B 5/11

(52) U.S. Cl. CPC A61B 5/067; G06F 3/017; G01N 29/4427; A61B 5/0816; A61B 5/681; A61B 5/11; A61B 2562/04

(57) ABSTRACT

Disclosed is a method of detecting a smoking behavior based on an acceleration sensor. A method of detecting a smoking behavior based on an acceleration sensor according to an exemplary embodiment of the present disclosure includes: calculating a wrist acceleration variation value which is a variation value of gravitational acceleration applied based on a gravitational direction with respect to a user's wrist in accordance with a motion of the user's wrist by using a three-axis acceleration sensor worn on the user's wrist; converting the wrist acceleration variation value into a wrist angle variation value which is a variation value of a wrist angle that indicates an angle to the user's wrist based on the gravitational direction; comparing at least one template signal made by modeling the variation value of the user's wrist angle corresponding to a smoking behavior with the wrist angle variation value; and detecting the user's smoking behavior based on the comparison result.

### CROSS-REFERENCE TO RELATED APPLICATIONS

This application claims the priority of Korean Patent Application No. 10-2016-0053304 filed on Apr. 29, 2016, in the Korean Intellectual Property Office, the disclosure of which is incorporated herein by reference.

### BACKGROUND

### Field

The present disclosure relates to detection of a user's behavior, and more particularly, to a method and an apparatus for detecting a user's smoking behavior.

### Description of the Related Art

As the well-being craze is spread globally, interests in personal health is being increased. In particularly, as the number of persons, who worry about deterioration in health due to smoking, is increased, researches for figuring out a personal smoking amount are being progressively conducted.

However, in the related art, there is a problem in that a plurality of sensors needs to be used to detect a personal smoking status, and there is also a problem in that accuracy deteriorates in a case in which the personal smoking status is detected by using a single sensor.

Therefore, there is a growing need for a technology capable of accurately detecting the personal smoking status even by using a single sensor.

As literature associated with the present disclosure, there is Japanese Patent Application Laid-Open No. 2013-162975 (entitled “Smoking Estimation Device”, published on Aug. 22, 2013).

### SUMMARY

An object to be achieved by the present disclosure is to provide a method and an apparatus for detecting a smoking behavior based on an acceleration sensor, which accurately detect a user's smoking behavior only by using the acceleration sensor.

According to an aspect of the present disclosure, there is provided a method of detecting a smoking behavior based on an acceleration sensor, the method including: calculating a wrist acceleration variation value which is a variation value of gravitational acceleration applied based on a gravitational direction with respect to a user's wrist in accordance with a motion of the user's wrist by using a three-axis acceleration sensor worn on the user's wrist; converting the wrist acceleration variation value into a wrist angle variation value which is a variation value of a wrist angle that indicates an angle to the user's wrist based on the gravitational direction; comparing at least one template signal made by modeling the variation value of the user's wrist angle corresponding to a smoking behavior with the wrist angle variation value; and detecting the user's smoking behavior based on the comparison result.

In particular, the calculating of the wrist acceleration variation value may be performed based on variation of an initial angle which is an angle between the gravitational direction and one of three sensor axes and is calculated in an initial state in which one of the three sensor axes, which are three axes of the acceleration sensor, is positioned toward a ground surface.

In particular, the calculating of the wrist acceleration variation value may include: measuring initial gravitational acceleration with respect to the three axes in an initial state in which one of the three sensor axes, which are three axes of the acceleration sensor, is positioned toward a ground surface; calculating an initial angle between the gravitational direction and one of the three sensor axes by using the measured initial gravitational acceleration with respect to the three axes; converting, by using the calculated initial angle, the gravitational acceleration, which is measured in real time at the three sensor axes, into converted gravitational acceleration which is gravitational acceleration with respect to three gravitational axes that are three axes using the gravitational direction as a reference axis; and calculating a variation value of the converted gravitational acceleration corresponding to the reference axis which varies in accordance with a motion of the user's wrist as the wrist acceleration variation value.

In particular, the calculating of the wrist acceleration variation value may include: reading initial gravitational acceleration with respect to the three axes which is measured and stored in advance in an initial state in which one of the three sensor axes, which are three axes of the acceleration sensor, is positioned toward a ground surface; calculating an initial angle between the gravitational direction and one of the three sensor axes by using the read initial gravitational acceleration with respect to the three axes; calculating, by using the calculated initial angle, converted gravitational acceleration which is gravitational acceleration with respect to three gravitational axes that are three axes using the gravitational direction as a reference axis based on the gravitational acceleration which is measured in real time at the three sensor axes; and calculating a variation value of the converted gravitational acceleration corresponding to the reference axis which varies in accordance with a motion of the user's wrist as the wrist acceleration variation value.

In particular, the converting of the wrist acceleration variation value into the wrist angle variation value may include: calculating a normalized wrist acceleration variation value by normalizing the wrist acceleration variation value; and converting the normalized wrist acceleration variation value into the wrist angle variation value by using an inverse function of cosine.

In particular, the comparing of the at least one template signal with the wrist angle variation value may include comparing first and second template signals made by modeling a variation value of the user's wrist angle corresponding to the smoking behavior with the wrist angle variation value, a magnitude of the first template signal may be set to alternately have a first angle value or a second angle value for each predetermined period, and a magnitude of the second template signal may be set to alternately have the first angle value or the second angle value for each period opposite to the period of the first template signal.

In particular, the first and second angle values may have a value between 45 degrees and 135 degrees.

In particular, the comparing of the at least one template signal with the wrist angle variation value may include: creating a first error signal which is an error between the first template signal and the wrist angle variation value; creating a second error signal which is an error between the second template signal and the wrist angle variation value; and comparing a final error signal, which is an error between the first error signal and the second error signal, with a final error threshold value.

In particular, the detecting of the user's smoking behavior may include: detecting an effective final error signal which is a final error signal that exceeds the final error threshold value among the final error signals; comparing the number of effective final error signals, which is detected for a predetermined period of time, with a number threshold value; and detecting the user's behavior for the predetermined period of time as the smoking behavior based on the comparison result.

In particular, the comparing of the at least one template signal with the wrist angle variation value may include: creating a first error signal which is an error between the first template signal and the wrist angle variation value; creating a second error signal which is an error between the second template signal and the wrist angle variation value; and comparing the first error signal with a first error threshold value and comparing the second error signal with a second error threshold value.

In particular, the detecting of the user's smoking behavior may include: detecting an effective wrist angle variation value which is a wrist angle variation value in which a magnitude of the first error signal is smaller than that of the first error threshold value and a magnitude of the second error signal is greater than that of the second error threshold value; comparing the number of the effective wrist angle variation values, which is detected for a predetermined period of time, with a number threshold value; and detecting the user's behavior for the predetermined period of time as the smoking behavior based on the comparison result.

According to another aspect of the present disclosure, there is provided an apparatus for detecting a smoking behavior based on an acceleration sensor, the apparatus including: a wrist acceleration calculating unit which calculates a wrist acceleration variation value which is a variation value of gravitational acceleration applied based on a gravitational direction with respect to a user's wrist in accordance with a motion of the user's wrist by using a three-axis acceleration sensor worn on the user's wrist; a wrist angle converting unit which converts the wrist acceleration variation value into a wrist angle variation value which is a variation value of a wrist angle that indicates an angle to the user's wrist based on the gravitational direction; a comparison unit which compares at least one template signal made by modeling the variation value of the user's wrist angle corresponding to a smoking behavior with the wrist angle variation value; and a behavior detecting unit which detects the user's smoking behavior based on the comparison result.

In particular, the wrist acceleration calculating unit may calculate the wrist acceleration variation value based on variation of an initial angle which is an angle between the gravitational direction and one of three sensor axes and is calculated in an initial state in which one of the three sensor axes, which are three axes of the acceleration sensor, is positioned toward a ground surface.

In particular, the wrist acceleration calculating unit may measure initial gravitational acceleration with respect to the three axes in an initial state in which one of the three sensor axes, which are three axes of the acceleration sensor, is positioned toward a ground surface, calculate the initial angle between the gravitational direction and one of the three sensor axes by using the measured initial gravitational acceleration with respect to the three axes, convert, by using the calculated initial angle, the gravitational acceleration, which is measured in real time at the three sensor axes, into converted gravitational acceleration which is gravitational acceleration with respect to three gravitational axes that are three axes using the gravitational direction as a reference axis, and calculate a variation value of the converted gravitational acceleration corresponding to the reference axis which varies in accordance with a motion of the user's wrist as the wrist acceleration variation value.

In particular, the wrist acceleration calculating unit may read initial gravitational acceleration with respect to the three axes which is measured and stored in advance in an initial state in which one of the three sensor axes, which are three axes of the acceleration sensor, is positioned toward a ground surface, calculate an initial angle between the gravitational direction and one of the three sensor axes by using the read initial gravitational acceleration with respect to the three axes, calculate, by using the calculated initial angle, converted gravitational acceleration which is gravitational acceleration with respect to three gravitational axes that are three axes using the gravitational direction as a reference axis based on the gravitational acceleration which is measured in real time at the three sensor axes, and calculate a variation value of the converted gravitational acceleration corresponding to the reference axis which varies in accordance with a motion of the user's wrist as the wrist acceleration variation value.

In particular, the wrist angle converting unit may calculate a normalized wrist acceleration variation value by normalizing the wrist acceleration variation value, and convert the normalized wrist acceleration variation value into the wrist angle variation value by using an inverse function of cosine.

In particular, the comparison unit may compare first and second template signals made by modeling a variation value of the user's wrist angle corresponding to the smoking behavior with the wrist angle variation value, a magnitude of the first template signal may be set to alternately have a first angle value or a second angle value for each predetermined period, and a magnitude of the second template signal may be set to alternately have the first angle value or the second angle value for each period opposite to the period of the first template signal.

In particular, the comparison unit may create a first error signal which is an error between the first template signal and the wrist angle variation value, create a second error signal which is an error between the second template signal and the wrist angle variation value, and compare a final error signal, which is an error between the first error signal and the second error signal, with a final error threshold value.

In particular, the behavior detecting unit may detect an effective final error signal which is a final error signal that exceeds the final error threshold value among the final error signals, compare the number of effective final error signals, which is detected for a predetermined period of time, with a number threshold value, and detect the user's behavior for the predetermined period of time as the smoking behavior based on the comparison result.

In particular, the comparison unit may create a first error signal which is an error between the first template signal and the wrist angle variation value, create a second error signal which is an error between the second template signal and the wrist angle variation value, and compare the first error signal with a first error threshold value and compares the second error signal with a second error threshold value.

In particular, the behavior detecting unit may detect an effective wrist angle variation value which is a wrist angle variation value in which a magnitude of the first error signal is smaller than that of the first error threshold value and a magnitude of the second error signal is greater than that of the second error threshold value, compare the number of the effective wrist angle variation values, which is detected for a predetermined period of time, with a number threshold value, and detect the user's behavior for the predetermined period of time as the smoking behavior based on the comparison result.

According to the exemplary embodiment of the present disclosure, it is possible to accurately detect the user's smoking behavior only by using the acceleration sensor.

According to another exemplary embodiment of the present disclosure, it is possible to clearly distinguish the smoking behavior from behaviors such as an eating behavior, a motion of brushing teeth, or a motion of brushing hairs which is similar to the smoking behavior.

According to still another exemplary embodiment of the present disclosure, it is possible to estimate a stress level of a user based on frequency of the user's smoking behavior by accurately detecting the user's smoking behavior.

### BRIEF DESCRIPTION OF THE DRAWINGS

The above and other aspects, features and other advantages of the present disclosure will be more clearly understood from the following detailed description taken in conjunction with the accompanying drawings, in which:

FIG. 1 is a flowchart for explaining a method of detecting a smoking behavior based on an acceleration sensor according to an exemplary embodiment of the present disclosure;

FIG. 2 is a flowchart for explaining a process of calculating a wrist acceleration variation value according to the exemplary embodiment of the present disclosure;

FIG. 3A is a view for explaining gravitational acceleration with respect to three sensor axes and gravitational acceleration with respect to three gravitational axes according to the exemplary embodiment of the present disclosure;

FIG. 3B is a view for explaining gravitational acceleration with respect to three sensor axes and gravitational acceleration with respect to three gravitational axes according to the exemplary embodiment of the present disclosure;

FIG. 4 is a view for explaining a wrist direction in accordance with a user's smoking operation;

FIG. 5 is a view for explaining a process of converting a wrist acceleration variation value into a wrist angle variation value;

FIG. 6 is a flowchart for explaining a process of comparing a template signal with the wrist angle variation value according to the exemplary embodiment of the present disclosure;

FIG. 7A is a view for explaining the template signal according to the exemplary embodiment of the present disclosure;

FIG. 7B is a view for explaining the template signal according to the exemplary embodiment of the present disclosure;

FIG. 8 is a view for explaining first and second error signals according to the exemplary embodiment of the present disclosure;

FIG. 9A is a view for explaining a final error signal according to the exemplary embodiment of the present disclosure;

FIG. 9B is a view for explaining a process of comparing the final error signal with a final error threshold value according to the exemplary embodiment of the present disclosure;

FIG. 10 is a flowchart for explaining a process of detecting a user's smoking behavior according to the exemplary embodiment of the present disclosure;

FIG. 11A is a view for explaining an effective final error signal according to the exemplary embodiment of the present disclosure;

FIG. 11B is a view for explaining a cumulative number of the effective final error signal according to the exemplary embodiment of the present disclosure;

FIG. 12 is a flowchart for explaining a process of comparing a template signal with a wrist angle variation value according to another exemplary embodiment of the present disclosure;

FIG. 13 is a flowchart for explaining a process of detecting a user's smoking behavior according to another exemplary embodiment of the present disclosure;

FIG. 14A is a view for explaining a smoking time estimating method according to the exemplary embodiment of the present disclosure;

FIG. 14B is a view for explaining a smoking time estimating method according to another exemplary embodiment of the present disclosure;

FIG. 15 is a view for explaining an apparatus for detecting a smoking behavior according to the exemplary embodiment of the present disclosure;

FIG. 16 is a view for explaining accuracy of the method of detecting a smoking behavior according to the exemplary embodiment of the present disclosure;

FIG. 17A is a view for explaining performance of the method of detecting a smoking behavior according to the exemplary embodiment of the present disclosure; and

FIG. 17B is a view for explaining performance of the method of detecting a smoking behavior according to the exemplary embodiment of the present disclosure.

### DETAILED DESCRIPTION OF THE PREFERRED EMBODIMENT

Since the present disclosure may be modified in various forms and include various exemplary embodiments, specific exemplary embodiments will be illustrated in the drawings and described in detail. However, the description is not intended to limit the present disclosure to the specific exemplary embodiments, and it is to be understood that all the changes, equivalents, and substitutions belonging to the spirit and technical scope of the present disclosure are included in the present disclosure. In describing the respective drawings, like reference numerals designate like constituent elements.

The terms such as “first”, “second”, “A”, “B” may be used to describe various constituent elements, but the constituent elements should not be limited by the terms. These terms are used only to distinguish one constituent element from another constituent element. For example, a first component may be named a second component, and similarly, the second component may also be named the first component, without departing from the scope of the present disclosure. The term “and/or” includes any and all combinations of a plurality of the associated listed items.

When one constituent element is described as being “connected” or “coupled” to another constituent element, it should be understood that one constituent element can be connected or coupled directly to another constituent element, and an intervening constituent element can also be present between the constituent elements. When one constituent element is described as being “connected directly to” or “coupled directly to” another constituent element, it should be understood that no intervening constituent element is present between the constituent elements.

Terms used in the present application are used only to describe specific exemplary embodiments, and are not intended to limit the present disclosure. Singular expressions used herein include plurals expressions unless they have definitely opposite meanings in the context. In the present application, it will be appreciated that terms “including” and “having” are intended to designate the existence of characteristics, numbers, steps, operations, constituent elements, and components described in the specification or a combination thereof, and do not exclude a possibility of the existence or addition of one or more other characteristics, numbers, steps, operations, constituent elements, and components, or a combination thereof in advance.

All terms used herein including technical or scientific terms have the same meanings as meanings which are generally understood by those skilled in the technical field to which the present disclosure pertains unless they are differently defined. Terms defined in a generally used dictionary shall be construed that they have meanings matching those in the context of a related art, and shall not be construed in ideal or excessively formal meanings unless they are clearly defined in the present application.

Hereinafter, exemplary embodiments according to the present disclosure will be described in detail with reference to the accompanying drawings.

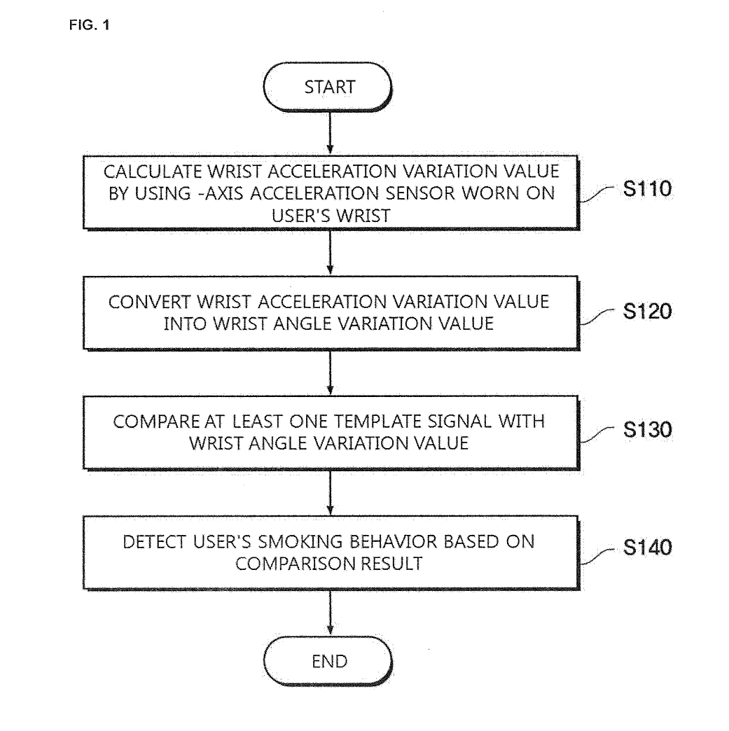

FIG. 1 is a flowchart for explaining a method of detecting a smoking behavior based on an acceleration sensor according to an exemplary embodiment of the present disclosure.

In step 110, by using a three-axis acceleration sensor worn on a user's wrist, an apparatus for detecting a smoking behavior calculates a wrist acceleration variation value which is an variation value of gravitational acceleration applied based on a gravitational direction with respect to the user's wrist in accordance with a motion of the user's wrist.

In this case, in a case in which the user lowers his/her wrist toward the ground surface during a smoking operation so that the wrist is positioned to coincide with the gravitational direction, the overall gravitational acceleration is applied to the user's wrist, and as a result, the wrist acceleration, which is the gravitational acceleration applied to the wrist, becomes 1 G. When the user slowly moves a cigarette toward the mouth in order to have the cigarette in the mouth, the gravitational acceleration applied to the wrist is also slowly decreased, the wrist acceleration is gradually decreased, and the wrist acceleration becomes a minus value from any moment. When the user stops the wrist operation in a state in which the user has the cigarette in the mouth, the minus gravitational acceleration value corresponding to the position of the wrist is maintained. As described above, in the present disclosure, the wrist acceleration value, which varies over time in accordance with the motion of the user's wrist, is defined as the wrist acceleration variation value, and the smoking operation based on the wrist acceleration variation value.

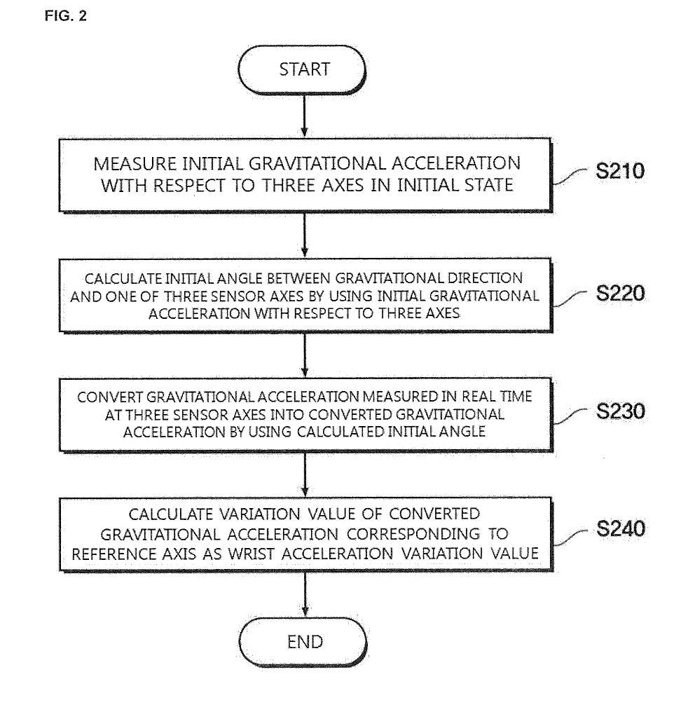

Meanwhile, the apparatus for detecting a smoking behavior may calculate the wrist acceleration variation value based on variation of an initial angle which is an angle between the gravitational direction and one of three sensor axes and is calculated in an initial state in which one of the three sensor axes, which are the three axes (x, y, and z axes) of the acceleration sensor, is positioned toward the ground surface. For example, in an initial state in which an x-axis direction of the three-axis acceleration sensor worn on the user's wrist is positioned toward the ground surface, an initial angle between the x-axis and the gravitational direction may be calculated, and the wrist acceleration variation value may be calculated based on variation of the calculated initial angle. A specific relevant operation will be described below with reference to FIG. 2.

In step 120, the apparatus for detecting a smoking behavior converts the wrist acceleration variation value into a wrist angle variation value which is a variation value of a wrist angle that indicates an angle to the user's wrist based on the gravitational direction.

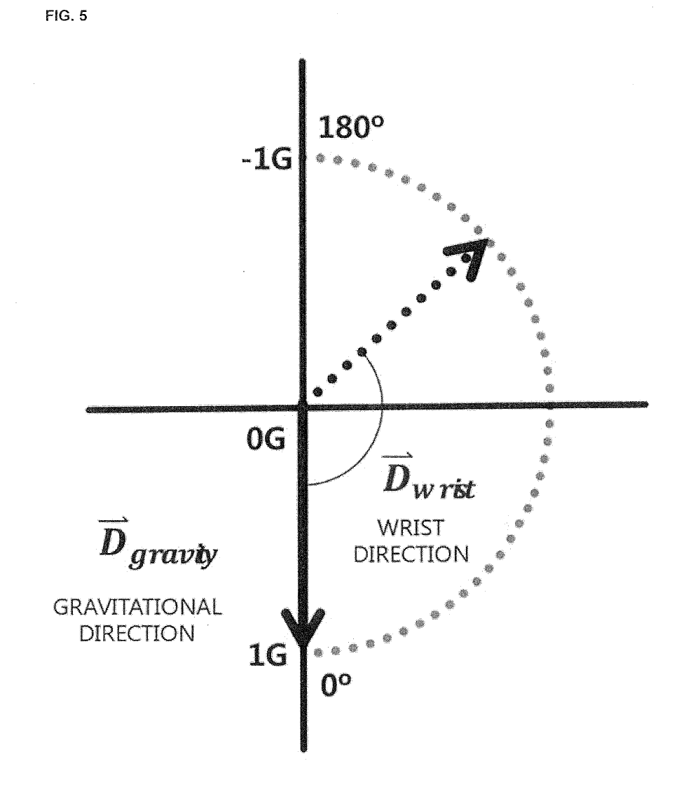

In this case, the apparatus for detecting a smoking behavior may calculate a normalized wrist acceleration variation value by normalizing the wrist acceleration variation value, and may convert the normalized wrist acceleration variation value into the wrist angle variation value by using an inverse function of cosine, and this process will be described below with reference to FIG. 5.

In step 130, the apparatus for detecting a smoking behavior compares a at least one template signal, which is made by modeling a variation value of the user's wrist angle corresponding to the smoking behavior, with the wrist angle variation value.

In this case, the apparatus for detecting a smoking behavior may compare a single template signal with the wrist angle variation value, but may compare two template signals with the wrist angle variation value.

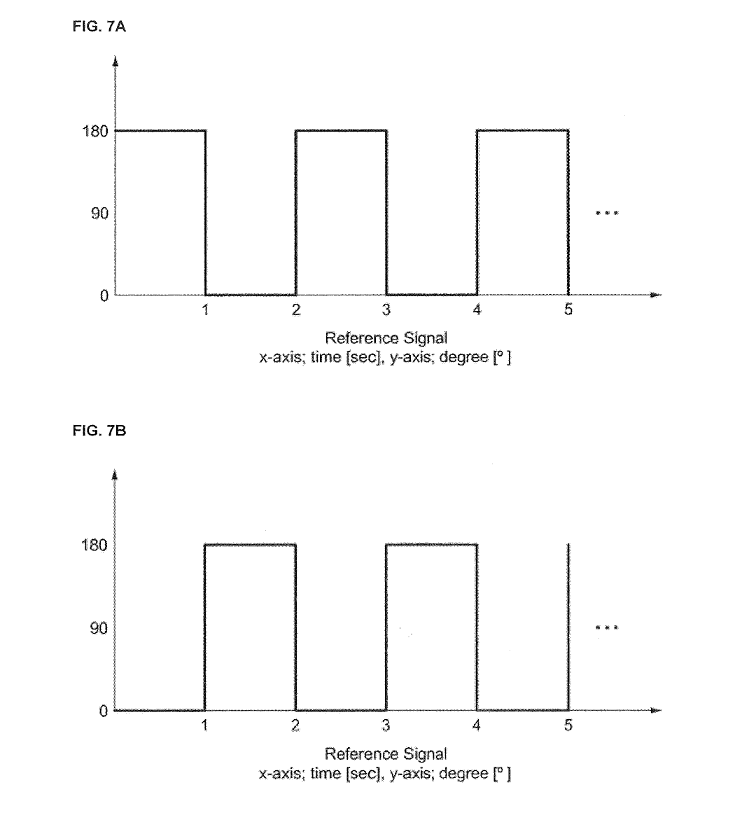

For example, the two template signals may be configured as illustrated in FIG. 7, and the two template signals will be described with reference to FIG. 7.

FIG. 7 is a view for explaining the template signal according to the exemplary embodiment of the present disclosure.

FIG. 7A illustrates a first template signal, FIG. 7B illustrates a second template signal, and it can be seen that the first template signal has a value of 180 degrees for each period of one second (0 to 1 second, 2 to 3 seconds, 4 to 5 seconds . . . ), and the second template signal has a value of 180 degrees for each period (1 to 2 seconds, 3 to 4 seconds . . . ) opposite to the period of the first template signal. The reason why the template signal is preset to have 0 degree or 180 degrees as described above is that the user's wrist angle is changed from the gravitational direction (0 degree) in a direction toward the face (180 degrees) during the smoking behavior.

Meanwhile, the first and second template signals illustrated in FIG. 7 are signals made by modeling the user's operation of raising his/her hand holding the cigarette toward his/her mouth and then lowering his/her hand toward the ground surface at an interval of approximately 1 second when the user performs the smoking behavior, and in another exemplary embodiment, the period may be adjusted to 0.5 second, 1.5 seconds, or the like.

Meanwhile, in FIG. 7, the first and second template signals are illustrated as having a size of 180 degrees, but particularly, the first and second template signals may have a value between 45 degrees and 135 degrees. The reason is that an angle of the wrist is generally an angle between 45 degrees and 135 degrees when the user smokes.

In step 140, the apparatus for detecting a smoking behavior detects the user's smoking behavior based on the comparison result.

In this case, the apparatus for detecting a smoking behavior determines that the user smokes in a case in which a result of comparing the template signal with the wrist angle variation value satisfies a predetermined condition.

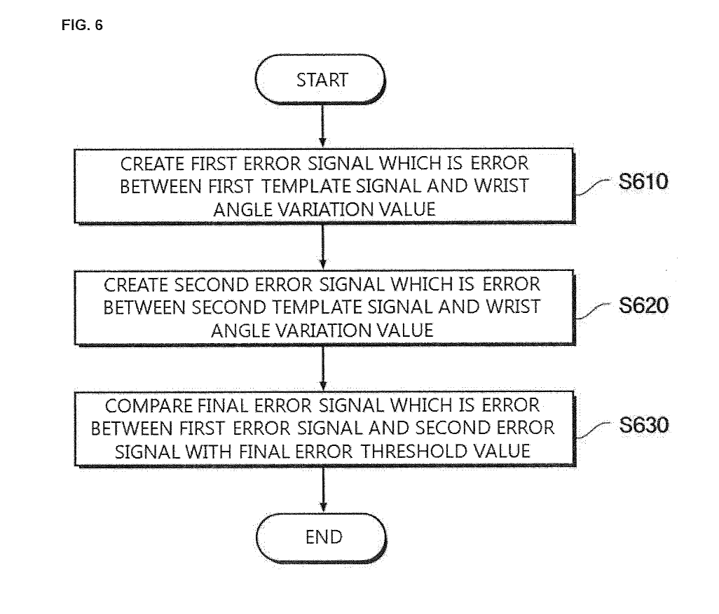

In a case in which the apparatus for detecting a smoking behavior compares the first and second template signals with the wrist angle variation value, the smoking behavior is detected when the wrist angle variation value is very similar to any one of the first and second template signals, and this configuration will be described below with reference to FIGS. 6 to 13.

In this case, to determine similarity between the template signal and the wrist angle variation value, algorithms such as relative (percent) accuracy, cross correlation, and convolution may be applied.

FIG. 2 is a flowchart for explaining a process of calculating the wrist acceleration variation value according to the exemplary embodiment of the present disclosure.

In step 210, the apparatus for detecting a smoking behavior measures initial gravitational acceleration with respect to the three axes in an initial state in which one of the three sensor axes, which are the three axes of the acceleration sensor, is positioned toward the ground surface.

For example, in an initial state in which the x-axis direction of the three-axis acceleration sensor worn on the user's wrist is positioned toward the ground surface, initial gravitational acceleration xᵢₙᵢ, Yᵢₙᵢ, and zᵢₙᵢ with respect to the three axes may be measured.

In another exemplary embodiment, it is possible to read the initial gravitational acceleration xᵢₙᵢ, yᵢₙᵢ, and zᵢₙᵢ with respect to the respective three axis, which is measured and stored in advance in the initial state in which one of the three sensor axes, which are the three axes of the acceleration sensor, is positioned toward the ground surface, instead of obtaining the initial gravitational acceleration by measuring the initial gravitational acceleration.

In step 220, the apparatus for detecting a smoking behavior may calculate the initial angle between the gravitational direction and one of the three sensor axes by using the measured initial gravitational acceleration with respect to the respective three axes.

More specifically, the apparatus for detecting a smoking behavior may calculate initial angles θ and ρ by using an inverse function of sine as described by Expression 1 in a case in which the initial angle between the x-axis and the gravitational direction is calculated in the initial state in which the x-axis direction of the three-axis acceleration sensor worn on the user's wrist is positioned toward the ground surface.

$$θ = {sin}^{- 1} ( \frac{{x}_{ini}}{\sqrt{{x}_{ini}^{2} + {y}_{ini}^{2}}} ) \\ ρ = {sin}^{- 1} ( \frac{{y}_{ini}}{\sqrt{{x}_{ini}^{2} + {y}_{ini}^{2} + {z}_{ini}^{2}}} ) \qquad [ Expression 1 ]$$

In this case, θ means a horizontal direction angle in a Cartesian coordinate system, and ρ means a vertical direction angle in the Cartesian coordinate system.

In another exemplary embodiment, in a case in which the apparatus for detecting a smoking behavior reads the initial gravitational acceleration stored in the apparatus for detecting a smoking behavior, the apparatus for detecting a smoking behavior may calculate the initial angle between the gravitational direction and one of the three sensor axes by using the read initial gravitational acceleration with respect to the three axes.

The reason why the initial angle between the gravitational direction and one of the three axes of the acceleration sensor is calculated as described above is to detect the motion of the user's wrist based on the gravitational direction, and in a case in which the initial angle based on the gravitational direction is not used, it is impossible to distinguish an operation of raising the wrist toward the user's lip from the ground surface and then lowering the wrist for smoking from an operation of straightening the user's wrist toward a front side of the body and bending the wrist.

In addition, even in a case in which the acceleration sensor may be worn on the user's wrist without distinguishing the left and right sides, it is possible to accurately detect the user's smoking behavior by calculating and utilizing the initial angle between the gravitational direction and one axis of the acceleration sensor.

In step 230, by using the calculated initial angle, the apparatus for detecting a smoking behavior converts the gravitational acceleration, which is measured in real time at the respective three sensor axes, into converted gravitational acceleration which is gravitational acceleration with respect to the three gravitational axes which are three axes defined by using the gravitational direction as a reference axis.

More specifically, the apparatus for detecting a smoking behavior may convert gravitational acceleration X, Y, and Z, which is measured in real time by the three-axis acceleration sensor, into converted gravitational acceleration X†, Y†, and Z† by using the initial angles θ and ρ as described by Expression 2.

$$[ \begin{matrix}cos θ & 0 & - sin θ \\ - sin θ sin ρ & cos ρ & - cos θ sin ρ \\ sin θ cos ρ & sin ρ & cos θ sin ρ\end{matrix} ] [ \begin{matrix}X \\ Y \\ Z\end{matrix} ] = [ \begin{matrix}{X}^{†} \\ {Y}^{†} \\ {Z}^{†}\end{matrix} ] \qquad [ Expression 2 ]$$

In this case, Z† is converted gravitational acceleration corresponding to the reference axis according to the gravitational direction, and X† and Y† are converted gravitational acceleration with respect to the axes orthogonal to Z†.

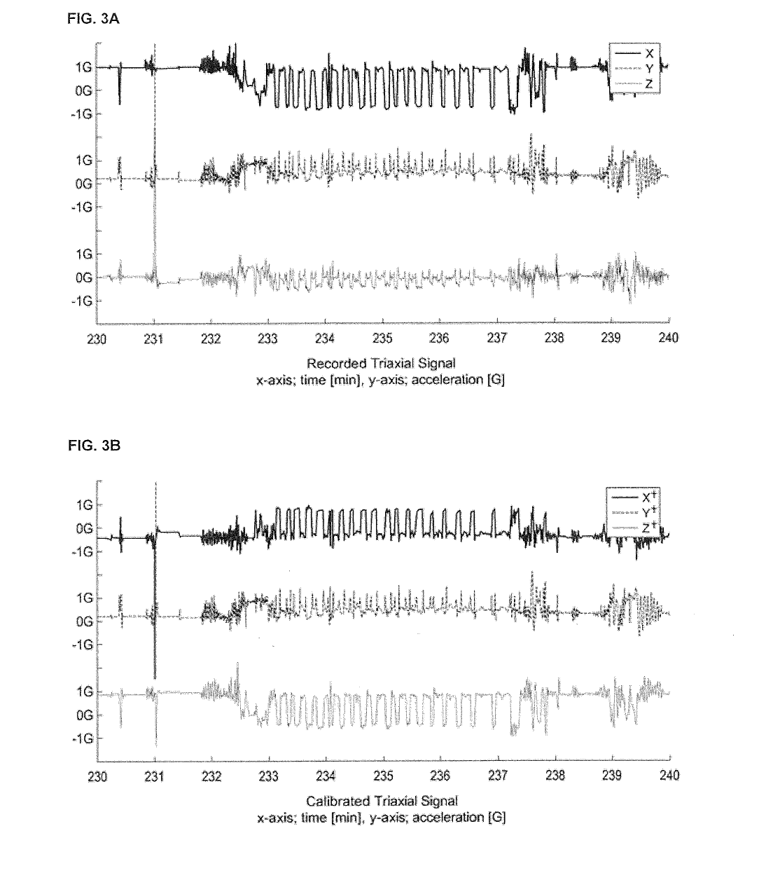

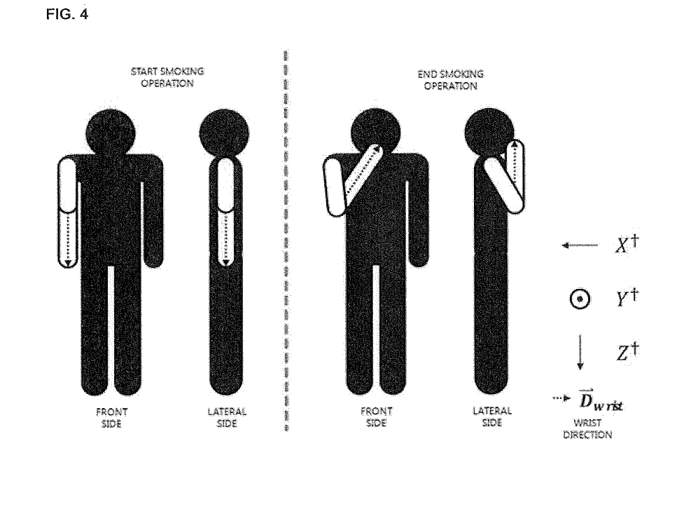

Now, a relationship between the gravitational acceleration X, Y, and Z and the converted gravitational acceleration X†, Y†, and Z† will be described with reference to FIGS. 3 and 4.

FIG. 3 is a view for explaining the gravitational acceleration with respect to the three sensor axes and the gravitational acceleration with respect to the three gravitational axes according to the exemplary embodiment of the present disclosure.

FIG. 3A is a view illustrating the gravitational acceleration with respect to the three sensor axes which illustrates the gravitational acceleration X, Y, and Z measured by the three-axis acceleration sensor while the user performs the smoking behavior, in which a horizontal axis indicates a time axis using the minute (min) as a unit, and a vertical axis indicates magnitudes of the gravitational acceleration using G as a unit. In the present disclosure, it is possible to convert the gravitational acceleration X, Y, and Z with respect to the three axes of the acceleration sensor, which is measured as described above, into the converted gravitational acceleration X†, Y†, and Z† by combining the gravitational acceleration X, Y, and Z by means of Expression 2.

FIG. 3B is a view illustrating the gravitational acceleration with respect to the three gravitational axes, which illustrates the converted gravitational acceleration X†, Y†, and Z† which converted and calculated from the gravitational acceleration X, Y, and Z which is measured while the user performs the smoking behavior. Referring to FIG. 3B, a magnitude of Z† is alternately shown by 1 G or −0.7 G, and in a state of the smoking behavior, the converted gravitational acceleration Z† corresponding to the reference axis has a variation value as described above.

FIG. 4 is a view for explaining the wrist direction in accordance with the user's smoking operation.

Referring to FIG. 4, when the user begins to perform the smoking operation, the user lowers the wrist toward the ground surface in the gravitational direction, and overall gravitational acceleration of 1 G is applied to the user's wrist.

In contrast, when the user ends the smoking operation (the user have a cigarette in his/her mouth), the user's wrist direction is obliquely placed in a direction toward the user's mouth, such that the gravitational acceleration of 1 G is not entirely applied to the user's wrist, gravitational acceleration smaller than 1 G is applied to the user's wrist.

In FIG. 4, the gravitational acceleration corresponding to the gravitational direction is Z†, the gravitational acceleration corresponding to the left and right direction orthogonal to the gravitational direction is X†, the gravitational acceleration corresponding to the remaining direction orthogonal to all of the gravitational direction and the left and right direction is Y†, and the user's wrist direction is D̅_{wrist}.

In step 240, the apparatus for detecting a smoking behavior calculates the wrist acceleration variation value based on a variation value of converted gravitational acceleration corresponding to the reference axis which varies in accordance with the motion of the user's wrist.

For example, the apparatus for detecting a smoking behavior may define Z†, which is the converted gravitational acceleration corresponding to the reference axis, as the wrist acceleration W which is the gravitational acceleration applied to the wrist, and may calculate a variation value of Z†, which is the converted gravitational acceleration, as a variation value of the wrist acceleration W.

Meanwhile, the apparatus for detecting a smoking behavior needs to satisfy the following assumptions when performing the operations of the present disclosure.

A first assumption is that the motion of the user's wrist when smoking needs to be independent of an initial direction of the acceleration sensor. This means that a direction of the acceleration sensor worn on the user's wrist should not affect the motion of the wrist, that is, the acceleration sensor should not affect the smoking behavior itself even though the three-axis acceleration sensor is worn on the wrist in any direction.

A second assumption is that the user's smoking operation needs to be performed in a state in which the user stands up.

The second assumption is that it is considered that there is no motion acceleration component in the user's smoking operation. The reason is that the motion of the wrist when smoking is too slow to consider the motion acceleration, and as a result, in the present disclosure, only the gravitational acceleration applied to the user's wrist is considered, and motion acceleration according to the motion of the wrist is not considered.

FIG. 5 is a view for explaining a process of converting the wrist acceleration variation value into the wrist angle variation value.

In FIG. 5, in a case in which the user lowers his/her wrist toward the ground surface during a smoking operation so that the wrist is positioned to coincide with the gravitational direction, the overall gravitational acceleration is applied to the user's wrist, and as a result, the wrist acceleration, which is the gravitational acceleration applied to the wrist, becomes 1 G. When the user moves a cigarette toward the mouth in order to have the cigarette in the mouth, the wrist acceleration is gradually decreased, and the wrist acceleration becomes a minus value from any moment. For example, an angle indicated by a dotted line in FIG. 5 is a wrist angle when the user has the cigarette in the mouth, and the wrist angle may be 135 degrees, and in this case, the gravitational acceleration may be −0.7 G.

Meanwhile, the apparatus for detecting a smoking behavior according to the exemplary embodiment of the present disclosure may convert the wrist acceleration into the wrist angle by using the inverse function of cosine as described in Expression 3 to Expression 5. In this case, because a domain of the inverse function of cosine is [−1, 1], it is necessary to adjust a unit of the wrist acceleration having an acceleration unit (m/s²), and Expression 3 to Expression 5 define the adjustment.

$${\vec{D}}_{wrist} = {cos}^{- 1} ( \frac{W}{9.8 m \text{/} {s}^{2}} ) \qquad [ Expression 3 ]$$

In Expression 3, W is divided by 9.8 m/s² in order to normalize the wrist acceleration when the wrist acceleration is measured by the unit of m/s², and the wrist angle may be calculated by substituting the normalized wrist acceleration to the inverse function of cosine.

$${\vec{D}}_{wrist} = {cos}^{- 1} ( \frac{W}{{2}^{R}} ) \qquad [ Expression 4 ]$$

In Expression 4, W is divided by resolution of the corresponding acceleration sensor in order to normalize the wrist acceleration when the digital system-based acceleration sensor measures the wrist acceleration by the unit of G, and the wrist angle may be calculated by substituting the normalized wrist acceleration to the inverse function of cosine. In this case, the higher the R value, the higher the resolution.

$${\vec{D}}_{wrist} = {cos}^{- 1} ( \frac{W}{\sqrt{{x}_{ini}^{2} + {y}_{ini}^{2} + {z}_{ini}^{2}}} ) \qquad [ Expression 5 ]$$

In Expression 5, the wrist angle may be calculated by normalizing the wrist acceleration by using the initial gravitational acceleration xᵢₙᵢ, yᵢₙᵢ, and zᵢₙᵢ and substituting the normalized wrist acceleration to the inverse function of cosine instead of normalizing the wrist acceleration by dividing W by 9.8 m/s² when the wrist acceleration is measured by the unit of m/s².

Meanwhile, in a case in which during a process of applying Expression 3 to Expression 5, a motion acceleration component is measured by the acceleration sensor and the wrist acceleration has a value exceeding [−1 G, 1 G], the apparatus for detecting a smoking behavior according to the exemplary embodiment of the present disclosure may calculate a wrist angle by considering −1 G as a minimum value, and 1 G as a maximum value.

FIG. 6 is a flowchart for explaining a process of comparing the template signal with the wrist angle variation value according to the exemplary embodiment of the present disclosure.

In step 610, the apparatus for detecting a smoking behavior creates a first error signal which is an error between the first template signal and the wrist angle variation value.

In this case, when calculating an error, residual sum of squares, root mean square error, least absolute deviation, and mean absolute error algorithms may be applied.

Hereinafter, in the present disclosure, an error is calculated by using the residual sum of squares algorithm. However, the present disclosure is not limited thereto.

In step 620, the apparatus for detecting a smoking behavior creates a second error signal which is an error between the second template signal and the wrist angle variation value.

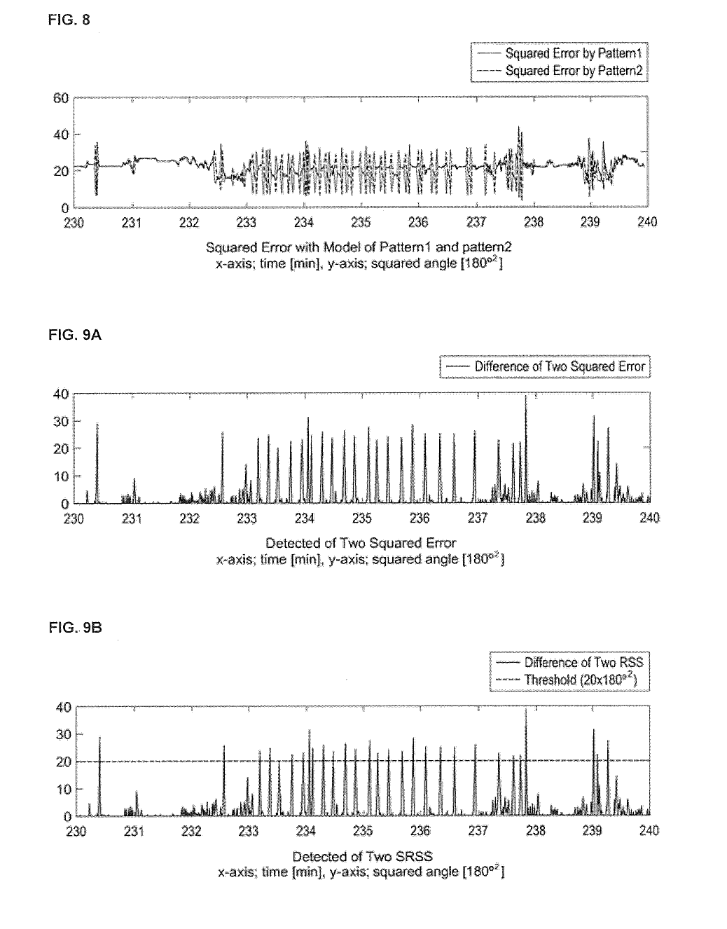

The first and second error signals will be described with reference to FIG. 8.

FIG. 8 is a view for explaining the first and second error signals according to the exemplary embodiment of the present disclosure, in which a signal indicated in blue indicates an example of the first error signal, a signal indicated in red indicates an example of the second error signal, a horizontal axis indicates time using the minute (min) as a unit, and a vertical axis indicates an error using a square of an angle as a unit.

In step 630, the apparatus for detecting a smoking behavior compares a final error signal, which is an error between the first error signal and the second error signal, with a final error threshold value.

The process of comparing the final error signal with the final error threshold value will be described with reference to FIG. 9.

FIG. 9A is a view for explaining the final error signal according to the exemplary embodiment of the present disclosure, FIG. 9B is a view for explaining the process of comparing the final error signal with the final error threshold value according to the exemplary embodiment of the present disclosure, FIG. 9A illustrates the final error signal which is an error between the first error signal and the second error signal, FIG. 9b illustrates the final error threshold value having a value of 20×180°2 by a red line, and the final error signal is compared with the final error threshold value indicated by a red line.

In the present disclosure, the reason why the two template signals are used as described above is to increase detection accuracy of the smoking behavior in comparison with a case in which a single template signal is used. That is, in a case in which a single template signal is used, a user's wrist acceleration variation value, which is not the smoking behavior, may be determined as the smoking behavior due to noise and the like, but in a case in which the two template signals are used, a likelihood of abnormal detection of the smoking behavior is relatively reduced.

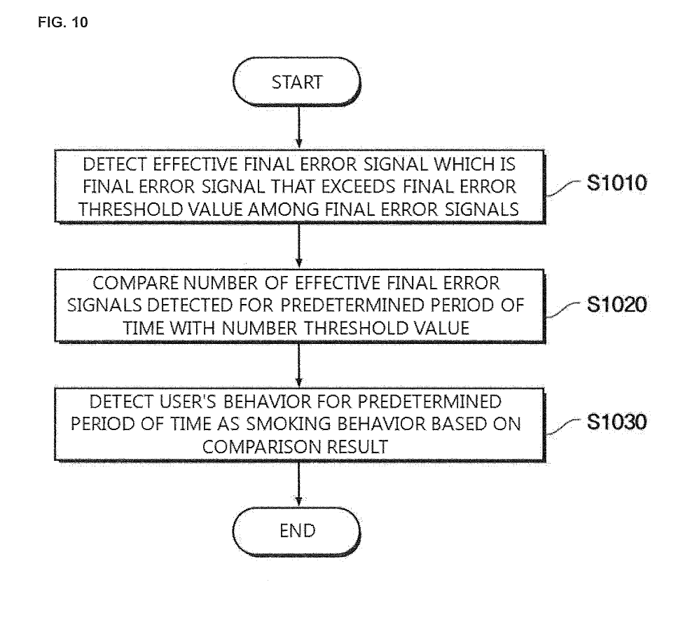

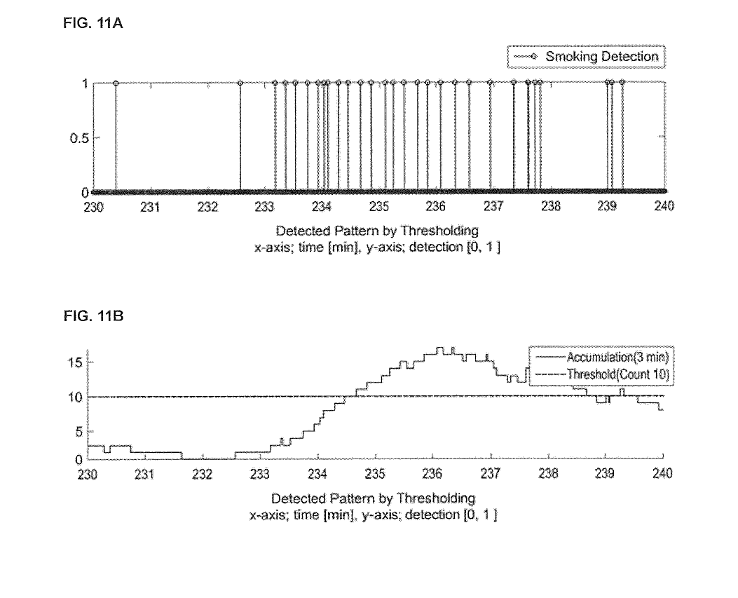

FIG. 10 is a flowchart for explaining a process of detecting the user's smoking behavior according to the exemplary embodiment of the present disclosure.

In step 1010, the apparatus for detecting a smoking behavior detects an effective final error signal which is a final error signal that exceeds the final error threshold value among the final error signals.

The effective final error signal will be described with reference to FIG. 11A. FIG. 11A is a view for explaining the effective final error signal according to the exemplary embodiment of the present disclosure, the effective final error signal, which is a final error signal that exceeds the final error threshold value among the final error signals, is set to have a magnitude of 1, and other signals are indicated by 0.

In step 1020, the apparatus for detecting a smoking behavior compares the number of effective final error signals, which is detected for a predetermined period of time, with a number threshold value.

The reason why the number of effective final error signals, which is detected for a predetermined period of time, is compared with the number threshold value as described above is to distinguish the smoking behaviors from other behaviors of moving the hand toward the periphery of the face, and the smoking behavior is assumed if the user raises the hand toward the face and lowers the hand ten or more times for three minutes.

In step 1030, based on the comparison result, the apparatus for detecting a smoking behavior detects the user's behavior for a predetermined period of time as the smoking behavior.

The process of comparing the number of effective final error signals with the number threshold value and the process of detecting the smoking behavior will be described with reference to FIG. 11B. FIG. 11B is a view for explaining a cumulative number of the effective final error signal according to the exemplary embodiment of the present disclosure, the cumulative number of the effective final error signal, which is detected for three minutes, is indicated in blue, the number threshold value is indicated by a red line, and the number of times is set to ten. Therefore, in FIG. 11B, a time section, which has a blue graph above the red line, may be considered as a time section in which the user smokes.

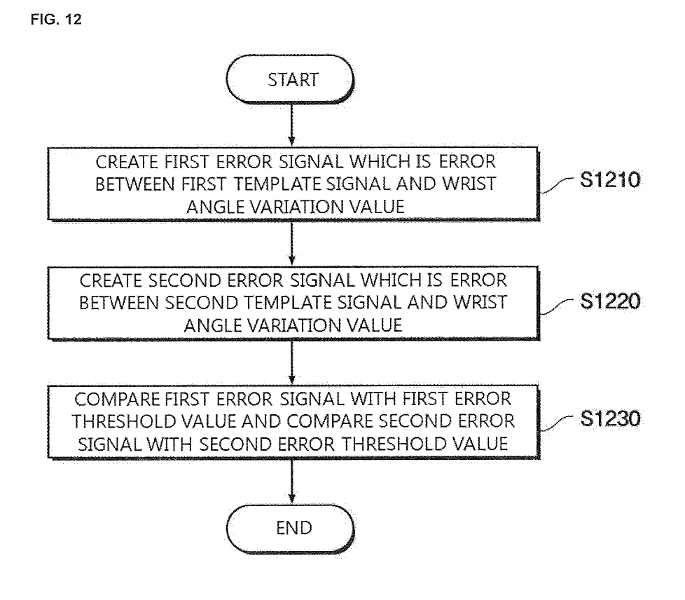

FIG. 12 is a flowchart for explaining a process of comparing a template signal with a wrist angle variation value according to another exemplary embodiment of the present disclosure.

In step 1210, the apparatus for detecting a smoking behavior creates a first error signal which is an error between the first template signal and the wrist angle variation value.

In step 1220, the apparatus for detecting a smoking behavior creates a second error signal which is an error between the second template signal and the wrist angle variation value.

In step 1230, the apparatus for detecting a smoking behavior compares the first error signal with a first error threshold value, and compares the second error signal with a second error threshold value.

The reason is to detect an effective wrist angle variation value which is a wrist angle variation value in which a magnitude of the first error signal is smaller than that of the first error threshold value and a magnitude of the second error signal is greater than that of the second error threshold value.

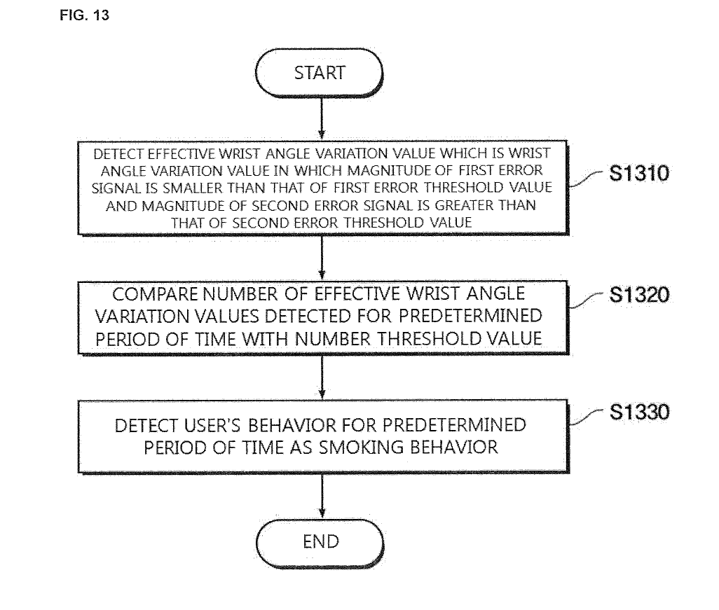

FIG. 13 is a flowchart for explaining a process of detecting a user's smoking behavior according to another exemplary embodiment of the present disclosure.

In step 1310, the apparatus for detecting a smoking behavior detects the effective wrist angle variation value which is a wrist angle variation value in which a magnitude of the first error signal is smaller than that of the first error threshold value and a magnitude of the second error signal is greater than that of the second error threshold value.

In step 1320, the apparatus for detecting a smoking behavior compares the number of effective wrist angle variation values, which is detected for a predetermined period of time, with the number threshold value.

In step 1330, based on the comparison result, the apparatus for detecting a smoking behavior detects the user's behavior for a predetermined period of time as the smoking behavior.

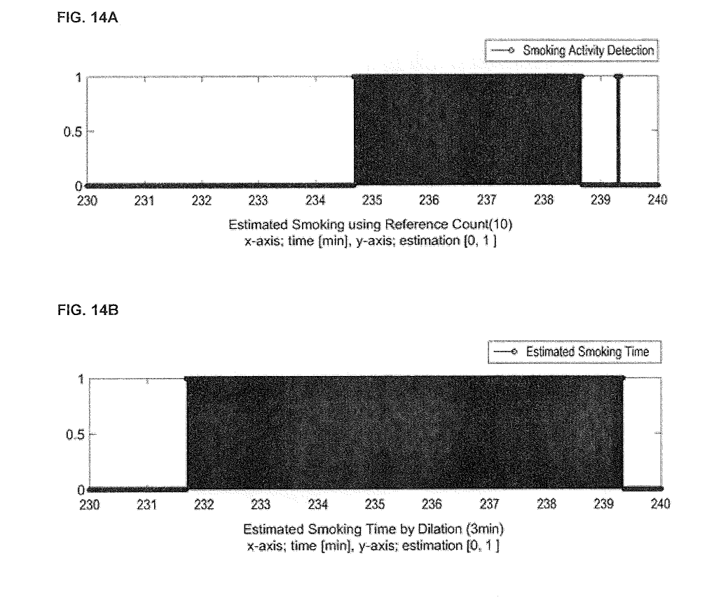

FIG. 14A is a view for explaining a smoking time estimating method according to the exemplary embodiment of the present disclosure.

Referring to FIG. 14A, all of the time section, in which the effective final error signal occurs ten or more times for three minutes in FIG. 11A, are estimated as a smoking time.

FIG. 14B is a view for explaining a smoking time estimating method according to another exemplary embodiment of the present disclosure.

FIG. 14B illustrates that by means of a method of estimating the smoking time by deducing the smoking time in respect to time before detecting the smoking behavior, the smoking time is deduced by applying a dilation technique the smoking behavior is considered before three minutes from a detection point in time because the smoking behavior is detected based on three minutes for which the cumulative number of the smoking behavior is calculated.

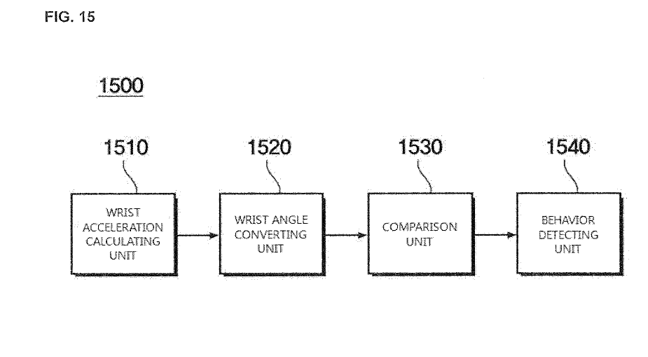

FIG. 15 is a view for explaining the apparatus for detecting a smoking behavior according to the exemplary embodiment of the present disclosure.

Referring to FIG. 15, the apparatus for detecting a smoking behavior according to the exemplary embodiment of the present disclosure includes a wrist acceleration calculating unit 1510, a wrist angle converting unit 1520, a comparison unit 1530, and a behavior detecting unit 1540.

By using the three-axis acceleration sensor worn on the user's wrist, the wrist acceleration calculating unit 1510 calculates the wrist acceleration variation value which is an variation value of gravitational acceleration applied based on the gravitational direction with respect to the user's wrist in accordance with the motion of the user's wrist.

The wrist angle converting unit 1520 converts the wrist acceleration variation value into the wrist angle variation value which is a variation value of a wrist angle that indicates an angle to the user's wrist based on the gravitational direction.

The comparison unit 1530 compares a at least one template signal, which is made by modeling a variation value of the user's wrist angle corresponding to the smoking behavior, with the wrist angle variation value.

The behavior detecting unit 1540 detects the user's smoking behavior based on the comparison result.

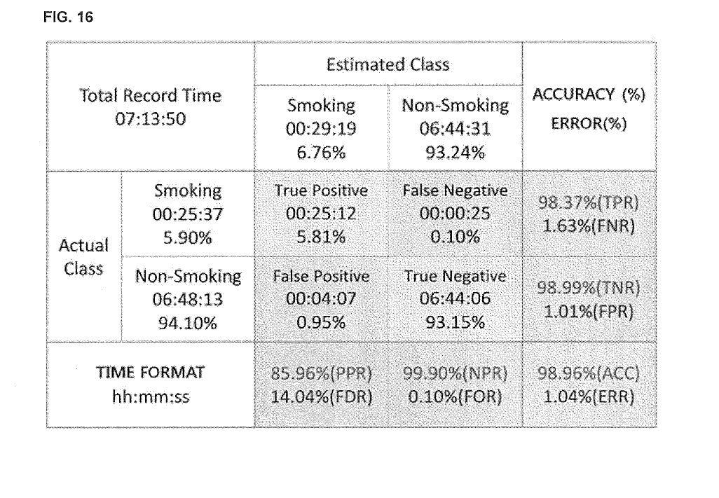

FIG. 16 is a view for explaining accuracy of the method of detecting a smoking behavior according to the exemplary embodiment of the present disclosure.

Referring to FIG. 16, as a result of comparing an actual behavior with a behavior estimated by the method of detecting a smoking behavior according to the present disclosure per second when an actual smoking time is about 29 minutes from data collected for about 7 hours, a TPR, which is a ratio made by estimating an actual smoking behavior as the smoking behavior, is 98.37%, a TNR, which is a ratio made by estimating an actual non-smoking behavior as the non-smoking behavior is 98.99%, and consequently, according to the present disclosure, accuracy is 98.96% in estimating the smoking behavior and the non-smoking behavior.

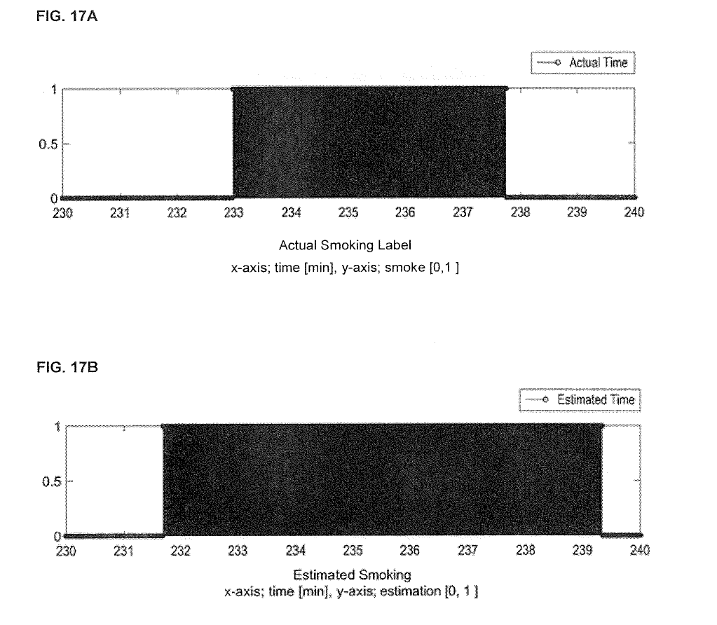

FIG. 17 is a view for explaining performance of the method of detecting a smoking behavior according to the exemplary embodiment of the present disclosure.

FIG. 17A is a view illustrating an actual smoking behavior time, FIG. 17B is a view illustrating a smoking behavior time estimated according to the exemplary embodiment of the present disclosure, and it can be seen that the overall actual smoking behavior time in FIG. 17A is included in the estimated smoking behavior time in FIG. 17B, and as a result, in the present disclosure, there is a great likelihood that the process of detecting the user's smoking behavior is not failed.

The present disclosure has been described above with reference to the exemplary embodiments. Those skilled in the art to which the present disclosure pertains will appreciate that various modifications are possible without departing from the essential characteristic of the present disclosure. Thus, it should be appreciated that the disclosed exemplary embodiments are intended to be illustrative, not restrictive. Here, it should be construed that the scope of the present disclosure is determined by the claims instead of the aforementioned description, and all differences within the equivalent scope thereto are included in the scope of the present disclosure.

What is claimed is:

1. A method of detecting a smoking behavior based on an acceleration sensor, the method comprising:
calculating a wrist acceleration variation value which is a variation value of gravitational acceleration applied based on a gravitational direction with respect to a user's wrist in accordance with a motion of the user's wrist by using a three-axis acceleration sensor worn on the user's wrist;
converting the wrist acceleration variation value into a wrist angle variation value which is a variation value of a wrist angle that indicates an angle to the user's wrist based on the gravitational direction;
comparing at least one template signal made by modeling the variation value of the user's wrist angle corresponding to a smoking behavior with the wrist angle variation value; and
detecting the user's smoking behavior based on the comparison result.

2. The method according to claim 1, wherein the calculating of the wrist acceleration variation value is performed based on variation of an initial angle which is an angle between the gravitational direction and one of three sensor axes and is calculated in an initial state in which one of the three sensor axes, which are three axes of the acceleration sensor, is positioned toward a ground surface.

3. The method according to claim 1, wherein the calculating of the wrist acceleration variation value includes:
measuring initial gravitational acceleration with respect to the three axes in an initial state in which one of the three sensor axes, which are three axes of the acceleration sensor, is positioned toward a ground surface;
calculating an initial angle between the gravitational direction and one of the three sensor axes by using the measured initial gravitational acceleration with respect to the three axes;
converting, by using the calculated initial angle, the gravitational acceleration, which is measured in real time at the three sensor axes, into converted gravitational acceleration which is gravitational acceleration with respect to three gravitational axes that are three axes using the gravitational direction as a reference axis; and
calculating a variation value of the converted gravitational acceleration corresponding to the reference axis which varies in accordance with a motion of the user's wrist as the wrist acceleration variation value.

4. The method according to claim 1, wherein the calculating of the wrist acceleration variation value includes:
reading initial gravitational acceleration with respect to the three axes which is measured and stored in advance in an initial state in which one of the three sensor axes, which are three axes of the acceleration sensor, is positioned toward a ground surface;
calculating an initial angle between the gravitational direction and one of the three sensor axes by using the read initial gravitational acceleration with respect to the three axes;
calculating, by using the calculated initial angle, converted gravitational acceleration which is gravitational acceleration with respect to three gravitational axes that are three axes using the gravitational direction as a reference axis based on the gravitational acceleration which is measured in real time at the three sensor axes; and
calculating a variation value of the converted gravitational acceleration corresponding to the reference axis which varies in accordance with a motion of the user's wrist as the wrist acceleration variation value.

5. The method according to claim 1, wherein the converting of the wrist acceleration variation value into the wrist angle variation value includes:
calculating a normalized wrist acceleration variation value by normalizing the wrist acceleration variation value; and
converting the normalized wrist acceleration variation value into the wrist angle variation value by using an inverse function of cosine.

6. The method according to claim 1, wherein the comparing of the at least one template signal with the wrist angle variation value includes comparing first and second template signals made by modeling a variation value of the user's wrist angle corresponding to the smoking behavior with the wrist angle variation value, a magnitude of the first template signal is set to alternately have a first angle value or a second angle value for each predetermined period, and a magnitude of the second template signal is set to alternately have the first angle value or the second angle value for each period opposite to the period of the first template signal.

7. The method according to claim 6, wherein the first and second angle values have a value between 45 degrees and 135 degrees.

8. The method according to claim 6, wherein the comparing of the at least one template signal with the wrist angle variation value includes:
creating a first error signal which is an error between the first template signal and the wrist angle variation value;
creating a second error signal which is an error between the second template signal and the wrist angle variation value; and
comparing a final error signal, which is an error between the first error signal and the second error signal, with a final error threshold value.

9. The method according to claim 8, wherein the detecting of the user's smoking behavior includes:
detecting an effective final error signal which is a final error signal that exceeds the final error threshold value among the final error signals;
comparing the number of effective final error signals, which is detected for a predetermined period of time, with a number threshold value; and
detecting the user's behavior for the predetermined period of time as the smoking behavior based on the comparison result.

10. The method according to claim 6, wherein the comparing of the at least one template signal with the wrist angle variation value includes:
creating a first error signal which is an error between the first template signal and the wrist angle variation value;
creating a second error signal which is an error between the second template signal and the wrist angle variation value; and
comparing the first error signal with a first error threshold value and comparing the second error signal with a second error threshold value.

11. The method according to claim 10, wherein the detecting of the user's smoking behavior includes:
detecting an effective wrist angle variation value which is a wrist angle variation value in which a magnitude of the first error signal is smaller than that of the first error threshold value and a magnitude of the second error signal is greater than that of the second error threshold value;
comparing the number of the effective wrist angle variation values, which is detected for a predetermined period of time, with a number threshold value; and
detecting the user's behavior for the predetermined period of time as the smoking behavior based on the comparison result.

12. An apparatus for detecting a smoking behavior based on an acceleration sensor, the apparatus comprising:
a wrist acceleration calculating unit which calculates a wrist acceleration variation value which is a variation value of gravitational acceleration applied based on a gravitational direction with respect to a user's wrist in accordance with a motion of the user's wrist by using a three-axis acceleration sensor worn on the user's wrist;
a wrist angle converting unit which converts the wrist acceleration variation value into a wrist angle variation value which is a variation value of a wrist angle that indicates an angle to the user's wrist based on the gravitational direction;
a comparison unit which compares at least one template signal made by modeling the variation value of the user's wrist angle corresponding to a smoking behavior with the wrist angle variation value; and
a behavior detecting unit which detects the user's smoking behavior based on the comparison result.

13. The apparatus according to claim 12, wherein the wrist acceleration calculating unit calculates the wrist acceleration variation value based on variation of an initial angle which is an angle between the gravitational direction and one of three sensor axes and is calculated in an initial state in which one of the three sensor axes, which are three axes of the acceleration sensor, is positioned toward a ground surface.

14. The apparatus according to claim 12, wherein the wrist acceleration calculating unit measures initial gravitational acceleration with respect to the three axes in an initial state in which one of the three sensor axes, which are three axes of the acceleration sensor, is positioned toward a ground surface, calculates the initial angle between the gravitational direction and one of the three sensor axes by using the measured initial gravitational acceleration with respect to the three axes, converts, by using the calculated initial angle, the gravitational acceleration, which is measured in real time at the three sensor axes, into converted gravitational acceleration which is gravitational acceleration with respect to three gravitational axes that are three axes using the gravitational direction as a reference axis, and calculates a variation value of the converted gravitational acceleration corresponding to the reference axis which varies in accordance with a motion of the user's wrist as the wrist acceleration variation value.

15. The apparatus according to claim 12, wherein the wrist acceleration calculating unit reads initial gravitational acceleration with respect to the three axes which is measured and stored in advance in an initial state in which one of the three sensor axes, which are three axes of the acceleration sensor, is positioned toward a ground surface, calculates an initial angle between the gravitational direction and one of the three sensor axes by using the read initial gravitational acceleration with respect to the three axes, calculates, by using the calculated initial angle, converted gravitational acceleration which is gravitational acceleration with respect to three gravitational axes that are three axes using the gravitational direction as a reference axis based on the gravitational acceleration which is measured in real time at the three sensor axes, and calculates a variation value of the converted gravitational acceleration corresponding to the reference axis which varies in accordance with a motion of the user's wrist as the wrist acceleration variation value.

16. The apparatus according to claim 12, wherein the wrist angle converting unit calculates a normalized wrist acceleration variation value by normalizing the wrist acceleration variation value, and converts the normalized wrist acceleration variation value into the wrist angle variation value by using an inverse function of cosine.

17. The apparatus according to claim 12, wherein the comparison unit compares first and second template signals made by modeling a variation value of the user's wrist angle corresponding to the smoking behavior with the wrist angle variation value, a magnitude of the first template signal is set to alternately have a first angle value or a second angle value for each predetermined period, and a magnitude of the second template signal is set to alternately have the first angle value or the second angle value for each period opposite to the period of the first template signal.

18. The apparatus according to claim 17, wherein the comparison unit creates a first error signal which is an error between the first template signal and the wrist angle variation value, creates a second error signal which is an error between the second template signal and the wrist angle variation value, and compares a final error signal, which is an error between the first error signal and the second error signal, with a final error threshold value.

19. The apparatus according to claim 18, wherein the behavior detecting unit detects an effective final error signal which is a final error signal that exceeds the final error threshold value among the final error signals, compares the number of effective final error signals, which is detected for a predetermined period of time, with a number threshold value, and detects the user's behavior for the predetermined period of time as the smoking behavior based on the comparison result.

20. The apparatus according to claim 17, wherein the comparison unit creates a first error signal which is an error between the first template signal and the wrist angle variation value, creates a second error signal which is an error between the second template signal and the wrist angle variation value, and compares the first error signal with a first error threshold value and compares the second error signal with a second error threshold value.

21. The apparatus according to claim 20, wherein the behavior detecting unit detects an effective wrist angle variation value which is a wrist angle variation value in which a magnitude of the first error signal is smaller than that of the first error threshold value and a magnitude of the second error signal is greater than that of the second error threshold value, compares the number of the effective wrist angle variation values, which is detected for a predetermined period of time, with a number threshold value, and detects the user's behavior for the predetermined period of time as the smoking behavior based on the comparison result.
